(19) United States
(12) Patent Application Publication
(10) Pub. No.: US 2007/0043290 A1
(43) Pub. Date: Feb. 22, 2007

(54) Method and apparatus for the detection of a bone fracture

(76) Inventors: Julius G. Goepp, Rochester, NY (US); Zachary M. Hoyt, Rochester, NY (US)

Correspondence Address: HOWARD J. GREENWALD P.C., 349 W. COMMERCIAL STREET SUITE 3075, EAST ROCHESTER, NY, 14445-2408, US

(21) Appl. No.: 11/496,952

(22) Filed: Aug. 1, 2006

Related U.S. Application Data
(60) Provisional application No. 60/704,990, filed on Aug. 3, 2005.

(51) Int. Cl. A61B 8/00

(52) U.S. Cl. 600/437

(57) ABSTRACT

Disclosed in this specification is a device configured to detect fractures in a bone by reflecting waves off of the bone. Certain parameters of the reflected wave are compared to a threshold condition. When the threshold condition is met, a first indication is generated. When the threshold condition is not met, a second indication is generated. This device allows detection of bone fractures without requiring that the user of the device be skilled in image interpretation (e.g. interpreting x-ray or ultrasound images).

### CROSS-REFERENCE TO RELATED PATENT APPLICATIONS

This application claims priority from applicant's co-pending patent application U.S. Ser. No. 60/704,990 (filed Aug. 2, 2005). The content of the aforementioned patent application is hereby incorporated by reference into this specification.

### FIELD OF THE INVENTION

This invention relates to ultrasound detection systems, more specifically to a short-range and inexpensive ultrasound system for layperson use in detecting bone and/or tissue irregularities in an injured limb that may have a fracture or other abnormality.

### BACKGROUND OF THE INVENTION

Hundreds of thousands of X-ray evaluations of injured bones are conducted each year in hospitals and clinics for the purpose of determining if a bone has been broken in an injury. The vast majority of these evaluations reveal normal bone, and the injury in such cases is labeled as a soft-tissue, usually trivial injury. In such cases, the X-ray evaluation was unnecessary. There is currently no reliable method for an accurate determination by a layperson of the likelihood that an injury involves a fracture. A device capable of delivering a simple “yes/no” signal regarding a predetermined, very high likelihood of a fracture would therefore potentially reduce unnecessary hospital visits, X-ray exposure, and costs.

Portable and relatively inexpensive non-X-ray diagnostic devices, such as ultrasound devices exist, but these either require expert training in the interpretation of the signal/image or are intended for single and specific purposes. For example, the single-purpose Doppler ultrasound device, the “SMART Needle,” is sold as a medical device for assistance in cannulating veins and avoiding arteries. Reference may be had to U.S. Pat. No. 5,259,385 to Miller (Apparatus for the cannulation of blood vessels), the contents of which are hereby incorporated by reference into this specification. This device contains a minute, disposable ultrasound transducer in the tip of the needle, and the signal is processed in a lightweight handheld unit. This device produces no diagnostic image, but simply provides an indication of proximity to pulsatile or non-pulsatile vessels. Other single-purpose, portable, and inexpensive ultrasound units are sold for layperson use, such as detecting and listening to fetal heart sounds, but such units are not intended for detecting abnormalities. While all of these devices are useful in their intended applications of providing information about soft tissue structure and function, the characteristics of ultrasound make it unsuitable for high-quality diagnostic images of bone. Thus, medical technology currently uses significantly more expensive, cumbersome, and potentially dangerous test methods, such as X-ray analysis, to identify acute structural changes in bone, such as those that appear in fractures or intrinsic bone lesions.

In many non-medical fields, ultrasound is used for the detection of hidden or buried objects covered with material(s) of different acoustic qualities than the object or material of interest. The devices exploit the differential reflection of sound waves from the interfaces between differing materials to provide a signal which is then processed to determine parameters such as depth or thickness of the object or material of interest. Ultrasound is used in the non-destructive testing (NDT) and detection of flaws in materials and structures at various and sometimes unknown depths. Reference may be had to U.S. Pat. No. 4,495,816 to Schlumberg (Process and System for Analyzing Discontinuities in Reasonably Homogeneous Medium); U.S. Pat. No. 6,022,318 to Koblanski (Ultrasonic Scanning Apparatus); U.S. Pat. No. 6,092,420 to Kimura (Ultrasonic Flaw Detector Apparatus and Ultrasonic Flaw-Detection Method); U.S. Pat. No. 6,585,652 to Lang (Measurement of Object Layer Thickness using Handheld Ultra-Sonic Devices and Methods Thereof); U.S. Pat. No. 6,588,278 to Takishita (Ultrasonic Inspection Device and Ultrasonic Probe); U.S. Pat. No. 6,606,909 to Dubois (Method and Apparatus to Conduct Ultrasonic Flaw Detection for Multi-Layered Structure); U.S. Pat. No. 6,640,632 to Katanaka (Ultrasonic Flaw Detection Method and Apparatus); U.S. Pat. No. 6,777,931 to Takada (Method of Displaying Signal Obtained by Measuring Probe and Device Therefore); and the like. Non-ultrasound devices are also available. See, for example, U.S. Pat. No. 5,457,394 to McEwan (Impulse Radar Studfinder); U.S. Pat. No. 5,893,102 to Maimone (Textual Database Management, Storage and Retrieval System Utilizing Word-Oriented, Dictionary-Based data Compression/Decompression); and the like. The content of each of the aforementioned patents is hereby incorporated by reference into this specification.

Other ultrasound devices have been used in medical diagnostic applications to examine soft tissues. Reference may be had to U.S. Pat. No. 4,080,860 to Goans (Ultrasonic Technique for Characterizing Skin Burns); U.S. Pat. No. 6,585,647 to Winder (Method and Means for Synthetic Structural Imaging and Volume Estimation of Biological Tissue Organs); U.S. Pat. No. 6,626,837 to Muramatsu (Ultrasonograph); U.S. Pat. No. 6,849,047 to Goodwin (Intraosteal Ultrasound During Surgical Implantation); U.S. Pat. No. 6,875,176 to Mourad (Systems and Methods for Making Noninvasive Physiological Assessments); U.S. patent application 2005/0033140A1 to de la Rosa (Medical Imaging Device and Method); 2005/01133691A1 to Liebschner (Noninvasive Tissue Assessment); and the like. The content of each of the aforementioned patents and patent applications is hereby incorporated by reference into this specification.

A number of prior art devices utilize ultrasound or electromagnetic energy to visualize or make determinations about certain properties of skeletal tissue, such as, for example, U.S. Pat. No. 4,421,119 Pratt (Apparatus for Establishing in Vivo Bone Strength); U.S. Pat. No. 4,476,873 to Sorenson (Ultrasound Scanning System for Skeletal Imaging); U.S. Pat. No. 4,655,228 to Shimura (Ultrasonic Diagnosis Apparatus for Tissue Characterization); U.S. Pat. No. 4,688,580 to Ko (Non-Invasive Electromagnetic Technique for Monitoring Bone Healing and Bone Fracture Localization); U.S. Pat. No. 4,754,763 to Doemland (Noninvasive System and Method for Testing the Integrity of an In Vivo Bone); U.S. Pat. No. 4,905,671 to Senge (Inducement of Bone Growth by Acoustic Shock Waves); U.S. Pat. No. 4,979,501 to Valchanov (Method and Apparatus for Medical Treatment of the Pathological State of Bones); U.S. Pat. No. 4,989,613 to Finkenberg (Diagnosis by Intrasound); U.S. Pat. No. 5,079,951 to Raymond (Ultrasonic Carcass Inspection); U.S. Pat. No. 5,235,981 to Hascoet (Use of Ultrasound for Detecting and Locating a Bony Region, Method and Apparatus for Detecting and Locating Such a Bony Region by Ultrasound); U.S. Pat. No. 5,309,898 to Kaufman (Ultrasonic Bone-Therapy and Assessment Apparatus and Method); U.S. Pat. No. 5,785,656 to Chiabrera (Ultrasonic Bone Assessment Method and Apparatus); U.S. Pat. No. 5,879,301 to Chiabrera (Ultrasonic Bone Assessment Method and Apparatus); U.S. Pat. No. 5,957,847 to Minakuchi (Method and Apparatus for Detecting Foreign Bodies in the Medullary Cavity); U.S. Pat. No. 6,299,524 to Janssen (Apparatus and Method for Detecting Bone Fracture in Slaughtered Animals, in Particular Fowl); U.S. Pat. No. 6,221,019 to Kantorovich (Ultrasonic Device for Determining Bone Characteristics); U.S. Pat. No. 6,322,507 to Passi (Ultrasonic Apparatus and Method for Evaluation of Bone Tissue); U.S. Pat. No. 6,585,651 to Nolte (Method and Device for Percutaneous Determination of Points Associated with the Surface of an Organ); U.S. Pat. No. 6,835,178 to Wilson (Ultrasonic Bone Testing with Copolymer Transducers); U.S. Pat. No. 6,899,680 to Hoff (Ultrasound Measurement Techniques for Bone Analysis); U.S. patent application 2004/0210135A1 to Hynynen (Shear Mode Diagnostic Ultrasound); and the like. The content of each of the aforementioned patents is hereby incorporated by reference into this specification.

Simple application of any of these existing technologies is inadequate for the purpose described herein. Human tissue varies greatly in the distance from skin to the underlying bone, and in the characteristics of the tissues between them. In order to achieve reliable tissue penetration and discrimination between normal and injured structures, and to eliminate noise in the signal, an operator of a prior art ultrasonic fracture detection device would need to be trained to control the depth and intensity of the scan, and to interpret the returned signal. This degree of complexity would make such a device cumbersome and unreliable. A need therefore exists for a simple, low-cost, handheld device capable of self-calibration; wherein the device is tolerant of a large degree of variability in user technique, and that is capable of producing a sensitive and specific indication of the likelihood of a fracture in the area of an injury.

Several prior art devices have been designed to incorporate features of ultrasonography into the determination of bone structure and condition in patients either at risk for or with known fractures or bone diseases, but to date, no approach has addressed the simple detection of previously unidentified fractures or other bone lesions. For example, U.S. Pat. No. 5,879,301 to Chiabrera (Ultrasonic Bone Assessment Method and Apparatus) discloses a method to test a bone to determine bone density. This is a useful technique for determining the degree of bone mineralization and degree of osteoporosis and hence, by implication, risk of future fracture, but it does not and is not intended to diagnose actual fracture in any bone. The teachings of Chiabrera are deficient in that they cannot be modified to detect existing bone fractures. Chiabrera relies upon testing an anatomical landmark, such as the edge of a heel bone, and transmitting ultrasonic waves through a bone. As is known to those skilled in the art, bone is relatively impervious to ultrasound. For example, and as disclosed in U.S. Pat. No. 4,655,228 to Shimura (Ultrasonic Diagnosis Apparatus for Tissue Characterization) ultrasonic diagnostic devices are generally adapted to observe differences in soft-tissue morphology and are unsuitable for use with bone.

Moreover, the invention of Chiabrera, as well as other prior art devices, are configured to generate complex diagnostic information for later interpretation by a qualified expert. To date, there is no device that permits the simple detection, as opposed to diagnosis, of a bone fracture by a layperson.

U.S. Pat. No. 5,235,981 to Hascoet (Use of Ultrasound for Detecting and Locating a Bony Region, Method and apparatus for Detecting and Locating such a Bony Region by Ultrasound) discloses an elaborate assembly which permits a skilled user to obtain detailed information about fracture location in three dimensions by using ultrasound, in cases in which the fracture is predetermined to exist.

The assembly of Hascoet is deficient in that it cannot be modified to be used by a layperson. The data provided by Hascoet must be interpreted by a qualified expert. Moreover the device of Hascoet cannot be modified to obtain a hand-held device, nor can it be used for primary detection of a suspected fracture.

The contents of U.S. Pat. Nos. 5,879,301; 4,655,228; and 5,235,981 are hereby incorporated by reference into this specification.

It is an object of the invention to provide an ultrasonic, handheld device that is configured for the primary detection of a suspected bone fracture, possible fracture or disease.

It is another object of the invention to provide a method for the primary detection of a suspected bone fracture, possible fracture or disease by ultrasound.

### SUMMARY OF THE INVENTION

In accordance with the present invention, there is provided a method and apparatus for detecting a bone fracture or disease using ultrasound. Within this specification, certain terms are given special meaning.

As used in this specification, the term ultrasound refers to a sonic wave with a frequency greater than the range of human hearing (typically about 20 KHz). As is known to those skilled in the art, sonic waves are distinguished from electromagnetic waves by their mode of propagation. Sonic waves require a medium, such as a solid, liquid, or gas, to travel through, whereas electromagnetic waves may travel through a vacuum.

The term transducer refers to a device that sends and receives wave signals. Examples of transducers include ultrasound transducers. One such ultrasound transducer is a transducer crystal which is a piezoelectric crystal that produces ultrasound in response to electrical stimulation, and produces electricity in response to stimulation by ultrasound energy.

As used in this specification, the term reflection refers to the redirection of a wave that occurs at the interface between two mediums with different acoustic properties. The region of reflection is significantly larger than the wavelength of the wave being used.

The term diagnostic ultrasound is the use of ultrasound to obtain graphic images for the purpose of making a medical diagnosis. A skilled user is required to interpret the graphic image that is obtained.

As used in this specification, the term detection ultrasound is the use of ultrasound to determine or predict the presence or absence of a physical condition of a structure. Detection ultrasound produces a binary display—the physical condition is either detected or it is not detected. A skilled user is not required to interpret the binary display that is produced.

The term depth refers to the distance along the axis defined by the direction of propagation of the wave from the center of the transducer face.

As used in this specification, the term electrical pulse or simply pulse refers to electrical impulses produced by an electrical pulse generator. The pulse may have the shape of a spike or of a square wave. Pulse amplitude is measured in volts or fractions thereof, pulse duration in seconds or fractions thereof, and pulse repetition frequency (PRF) is measured in pulses per second.

The term signal refers to the collective characteristics of the wave energy produced by or received at the face of the transducer in response to an electrical pulse delivered to the transducer or to a returning wave arriving at the face of the transducer. Signals have specific signal characteristics that include sound intensity, frequency, power spectrum, time(s) of flight, and others.

The term intensity (J) refers to the power per unit area at any specific distance from the transducer face or from a reflecting surface. Unlike power, which is solely dependent on emitter characteristics, intensity varies as the inverse square of the distance from the transducer. As used in this specification the terms reflected, received or echo intensity refer to the intensity of the echo received at the face of the transducer.

As used in this specification, the term intensity level (L_{J}) refers to the log₁₀ of the ratio of the received wave intensity to a predetermined standard intensity. The resulting dimensionless ratio is conventionally expressed in dB.

The term frequency refers to the frequency of the wave produced by the transducer, reflected from tissue interfaces, and received by the transducer. Frequency of ultrasound is measured in MHz. It is a characteristic of ultrasound transducer crystals to vibrate at a “center frequency” which corresponds to the crystal's natural resonant frequency. It will be understood by those skilled in the art that the vibrating crystal also produces ultrasound waves at frequencies above and below the center frequency. The center frequency and the other associated frequencies are reflected in varying amplitudes at each tissue interface. As used in this specification, the terms ultrasound frequency spectrum or ultrasound spectrum refer to the range of frequencies produced by the vibrating crystal during emission, or received by the transducer during reception. As used in this specification, the adjectives emitted, reflected, and received are used to identify the ultrasound frequency or spectrum under consideration.

As used in this specification the term power spectrum refers to the spectrum of sound power (at the emitter) or intensity (at the receiver) at each frequency over the range of frequencies contained in the emitted or received wave signal. Because the area of the transducer face is constant, the power spectra of the emitted and received signals can be directly compared in terms of either power or intensity.

The term beam refers to the beam of wave energy emitted by the transducer. As with any beam of wave energy, an ultrasound beam can be focused by appropriate lenses placed behind the source of energy or between the source of energy and a focal point. Although in physical space much of the beam inevitably spreads in a spherical fashion, the focal point and the center point of the transducer face define a straight line. As used in this specification, the direction, angle, or orientation of the ultrasound beam refers to the direction, angle, or orientation of the line between the center point of the transducer face and the focal point of the beam in relationship to an external object. In this specification that external object is the surface of an avian or mammalian bone.

As used in this specification, the term ultrasound echo refers to the ultrasound signal that is received at the transducer face after reflection or back-scattering from tissue interfaces, including the interface between soft tissue and bone. Ultrasound echoes have all of the same kinds of signal characteristics such as intensity, frequency, power spectrum, and others that are used to describe the original emitted signal. The actual values of these characteristics of the echo are of course different from the corresponding values for the emitted signal.

The term time of flight or TOF refers to the time elapsed between the emission of an ultrasound signal by the transducer and the arrival of the echo of that signal at the transducer face. Because the transducer itself is incapable of measuring time, and because the speed of light is large compared with the speed of sound in human tissue, the TOF that is measured by the processor will actually be the time between the generation of the electrical pulse that initiates the ultrasound signal and the arrival at the processor of the electrical signal that corresponds to the arrival of the echo of that ultrasound signal. It is apparent that other means for measuring TOF are not excluded by this definition.

As used in this specification the term electrical signal refers to the time-varying voltage and current fluctuations that are produced by the transducer crystal in response to the sound energy of the ultrasound echo arriving at the transducer face. This electrical signal produces a time-dependent waveform with similar characteristics to those of the ultrasound signal, such as amplitude, frequency, power spectrum, time of flight, and others.

The term amplitude of the electrical signal, measured in volts or amperes or fractions thereof, is directly proportional to the sound intensity of the received echo at the transducer face. As is known to one skilled in the art, the sound intensity level in dB can therefore also be calculated directly from the amplitude of the electrical signal produced by ultrasound at the transducer.

As used in this specification the terms frequency or frequencies of the electrical signal, measured in MHz, are substantially similar to the frequency or frequencies of the ultrasound echo signal received at the transducer face. As used in this specification, the term power spectrum of the electrical signal refers to the spectrum over all frequencies of the electrical signal amplitude associated with each frequency. It will be understood by a person skilled in the art that the frequencies and power spectra of the electrical signals are substantially similar to those of the ultrasound signal that produced them.

The term mathematical operations performed by the processor refers to such operations performed on the electrical signal(s) received by the processor from the signal processor or directly from the ultrasound transducer.

As used in this specification, the term Fourier transform refers to a mathematical operation that results in the decomposition of a time series signal into harmonics of different frequencies and amplitudes. The Fourier transform itself is a substantially lengthy calculation to compute when analyzing real-time signals. For that reason, as used in this specification, the Fast Fourier Transform FFT refers to a simpler calculation which is substantially advantageous. FFT allows a sequence of time-domain samples to be efficiently converted into a frequency representation using a previously-specified discrete time window. The FFT generates the frequency power spectra, allowing the processor to monitor the relative magnitudes of various components of a signal under inspection. The processed signal may be exploited over time to detect small changes in the frequency content of the real-time signals that correspond on the one hand to normal structures and on the other to fractures and bone diseases.

The discrete Gabor transform refers to a mathematical operation that produces a three-dimensional plot of signal intensity level (Lj) versus frequency and time. The discrete Gabor transform affords an additional means of identifying small frequency changes over time.

As used in this specification, the discrete Zak transform refers to a mathematical operation that can be used in combination with the discrete Fourier transform in a sum-of-products method to represent the discrete Gabor transform. As is known to one skilled in the art, many other mathematical operations consisting of transforms, discrete transforms, and any combinations thereof can be utilized to produce a processed signal that a processor can utilize to extract unique signal characteristics from raw signal information consisting of at least one of time, frequency, phase, and relative intensity.

The techniques described herein are advantageous because they are inexpensive and significantly more simple compared to prior art approaches. The techniques described herein are also advantageous because they increase the likelihood of detecting a true fracture (enhanced sensitivity) and decrease the likelihood of a false-positive identification (enhanced specificity), compared with prior art approaches. Additionally, the techniques of the invention are advantageous because they provide a range of alternatives, each of which is useful in appropriate situations and which may be used to cross-check one another for accuracy.

### BRIEF DESCRIPTION OF THE DRAWINGS

The invention will be described by reference to the following drawings, in which like numerals refer to like elements, and in which:

FIGS. 1A and 1B are perspective view and exploded views of one embodiment of the device of the present invention;

FIG. 1C is a perspective and exploded view of one footplate for use with the present invention;

FIG. 1D is a perspective view of another embodiment one device of the present invention;

FIG. 1E is an exploded view of the device illustrated in FIG. 1D;

FIG. 1F illustrates a perspective and exploded view of another device of the present invention;

FIG. 2 is a flow diagram of one process of the invention;

FIG. 3 is a flow diagram of the steps involved in the execution of step 204 of FIG. 2;

FIG. 3A is a graph depicting detection of bone by observation of the received signal intensity level;

FIG. 4 is a flow diagram of the steps involved in the execution of step 206 of FIG. 2;

FIG. 5 is a schematic view of the detection of a bone abnormality;

FIG. 6 is a schematic view of a bone abnormality and the resulting signal;

FIG. 7 is a schematic view of a second bone abnormality and the resulting signal;

FIG. 8 is a graphical representation of the signals from FIG. 6 and FIG. 7, and the resulting processed signal;

FIG. 9 is a schematic of one display of the invention and a corresponding signal graph;

FIG. 10 is a block diagram of one device of the present invention;

FIG. 11 is a block diagram of another device of the present invention;

FIG. 12 is a schematic view of another detection process;

FIG. 13 is yet another schematic view of a detection process of the present invention,

FIG. 14 is an illustration of the signals resulting from one experimental example of the present invention; and

FIG. 15 is a depiction of one assembly for use with the instant invention.

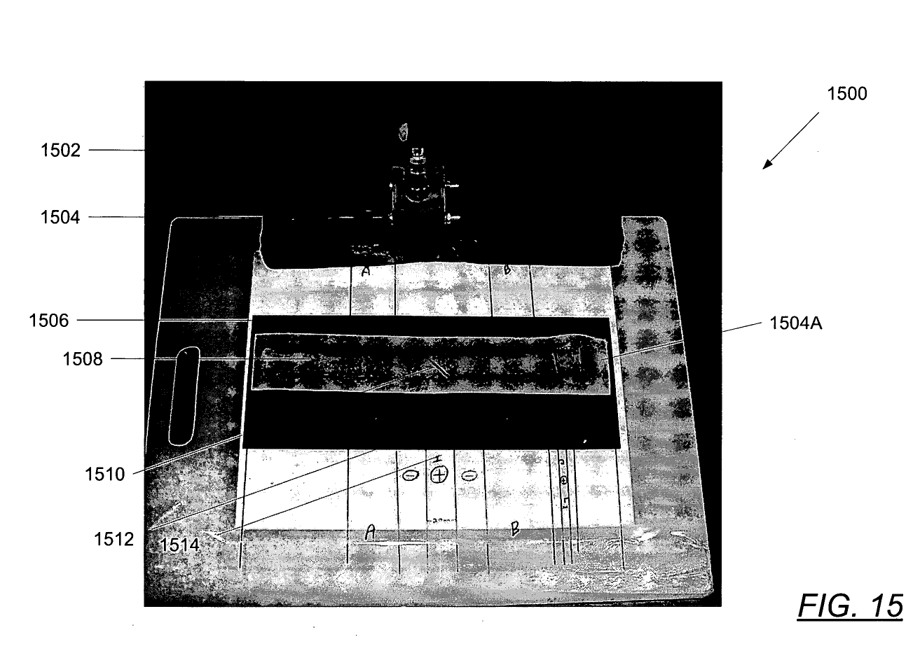

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention.

### DESCRIPTION OF THE PREFERRED EMBODIMENTS

For a general understanding of the present invention, reference is made to the drawings. In the drawings, like reference numerals have been used throughout to designate identical elements.

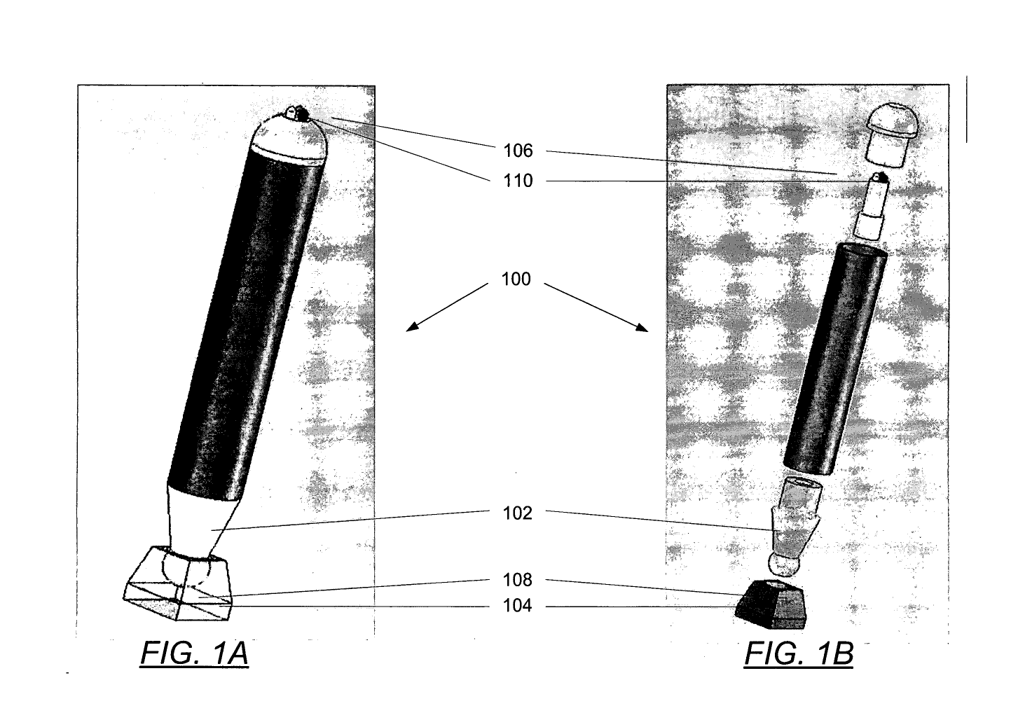

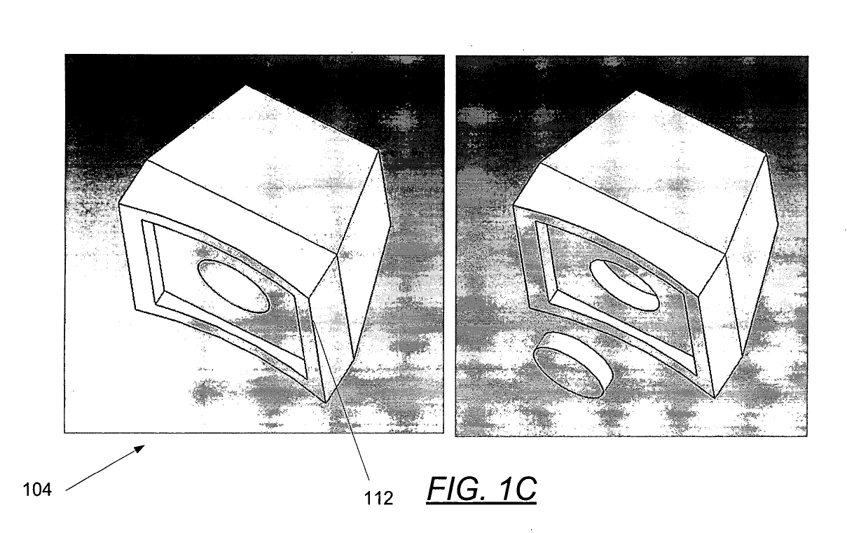

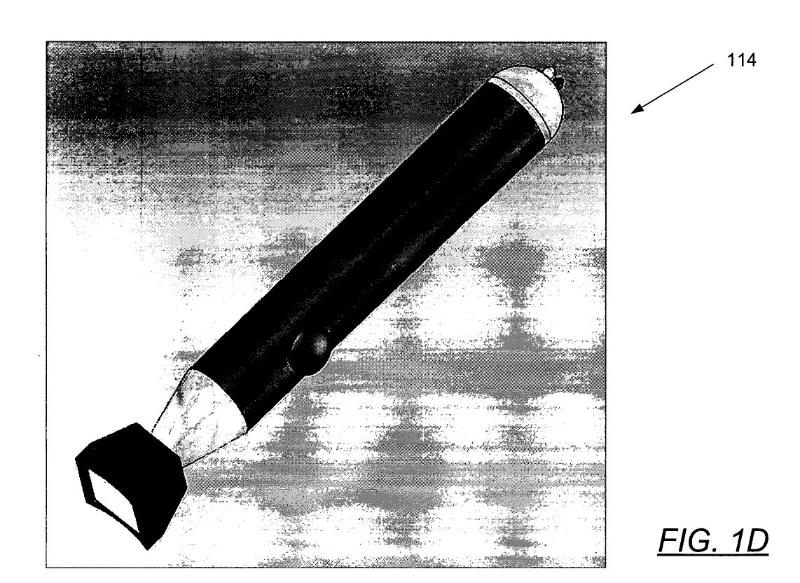

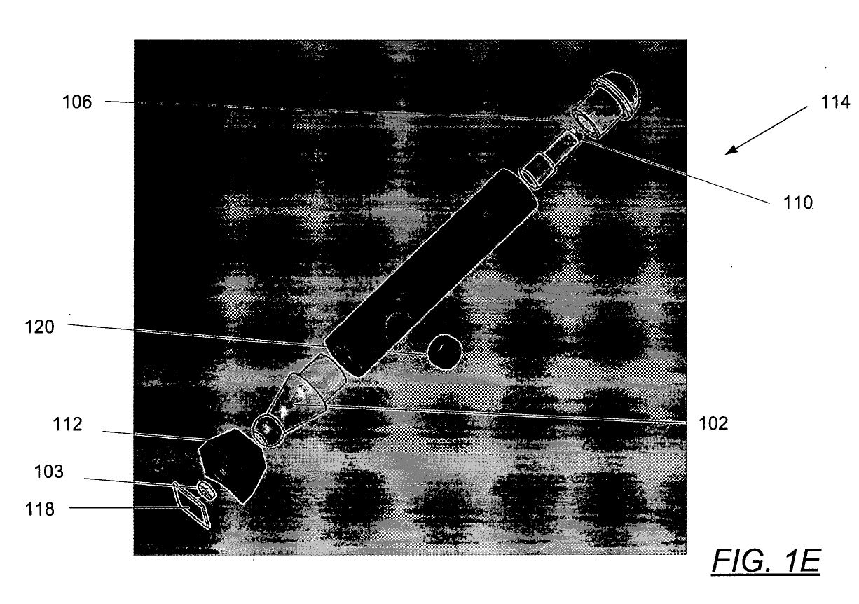

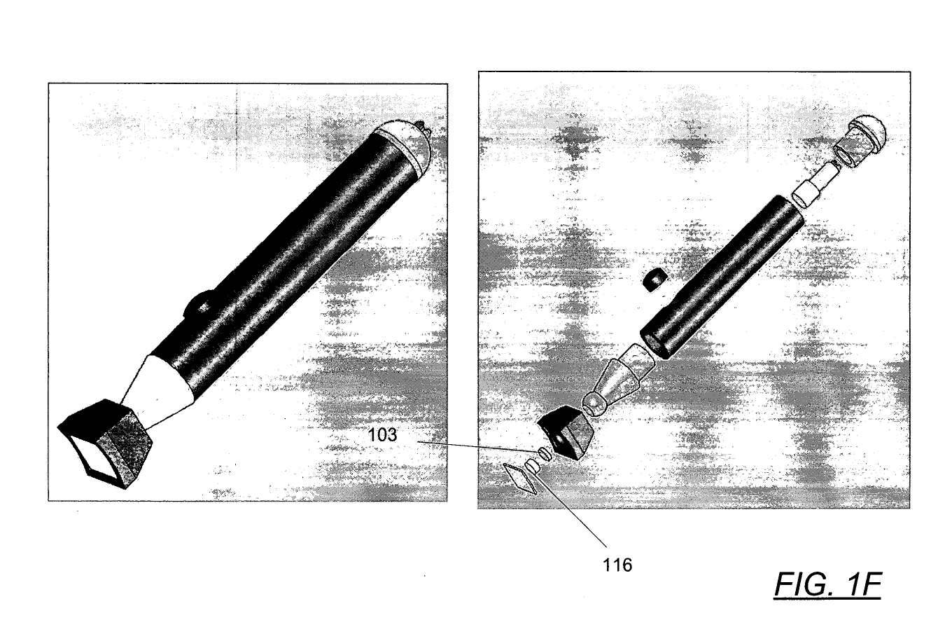

FIGS. 1A and 1B are schematic diagrams of one device of the present invention. In the embodiment depicted in FIGS. 1A and 1B, device 100 is comprised of probe 102, footplate 104, coupling medium 108, a processor (not shown), and display 106. Probe 102 is comprised of transducer 103 (see FIG. 1E). In the embodiment depicted in FIGS. 1A and 1B, footplate 104 is configured to be placed on a substantially flat surface. In the embodiment depicted in FIGS. 1A and 1B the transducer 103 is housed within the footplate 104. In the embodiment depicted, transducer 103 is configured to produce waves and thereafter receive the reflected waves when they are reflected off of a surface. In one embodiment, this surface is a bone.

It is desirable that the device be portable and hand-held, and be properly balanced so as not to induce any wobbling as the operator uses it. In one embodiment, a means of stabilization is provided that maintains a substantially steady and relatively light pressure of transducer 103 housed in footplate 104 against the skin. In one embodiment such means is comprised of small springs (not shown). In another embodiment, such means is comprised of shock absorbers. It is advantageous that the device has a minimal number of operator-dependent controls such as switches, and that it have a simple and intuitive display that is capable of informing the operator of a small number of conditions, such as adequate signal, poor signal, signal strength (to allow continued optimum positioning), and, of course, detection of an anomaly consistent with a fracture or bone disease. In another embodiment, the means of stabilization is a phased ultrasound array. One phased array suitable for use with the present invention is disclosed in U.S. Pat. No. 5,997,479 to Savord (Phased array acoustic systems with intra-group processors). Other phased array systems would be apparent to one skilled in the art. A phased ultrasound array is a series of ultrasonic transducers that are activated in series. When such a phased ultrasound array is used, measurements may be taken without moving the apparatus by selectively activating the transducers in a predetermined order.

In one embodiment, the footplate 104 is spring-mounted or otherwise equipped to provide a constant pressure against the skin. In one embodiment, the footplate 104 is comprised of means to measure the distance the footplate has traveled across the skin. In another embodiment, footplate 104 is comprised of means for measuring the pressure applied by the device to the skin. In one such embodiment, the processor is programmed to recognize a maximum pressure value, and causes a warning tone to be emitted by an audible sound generator, or “overpressure alarm,” if the user exceeds the maximum pressure value. In one embodiment, illustrated in FIG. 1C, footplate 104 is slightly concave on the side 112 facing the bone, which promotes alignment and stability of the footplate during motion along the scanning direction. In another embodiment, the footplate is comprised of a straight line indicator on its top (visible) surface which the user will align visually and by palpation with the apparent long axis of the bone.

Referring again to FIGS. 1A and 1B, and in the embodiment depicted therein, transducer 103 is configured to generate ultrasound waves with a frequency of from about 0.5 to about 50 MHz. In another embodiment, transducer 103 is configured to generate ultrasound waves with a frequency of from about 1 to about 20 MHz. In yet another embodiment, the frequency is from about 2 to about 12 MHz. A variety of ultrasound transducers are known to those skilled in the art. For example, a device capable of performing the methods disclosed herein would incorporate at least one ultrasound transducer selected from the group consisting of a single crystal transducer, a dual-element transducer, an array of multiple transducers, and combinations thereof. Reference may be had to The Biomedical Engineering Handbook (1995 CRC Press LLC, Joseph Bronzino Ed.) at pages 1077-1118, the contents of which are hereby incorporated by reference into this specification. Further reference may be had to U.S. Pat. No. 5,298,602 to Shikinami (Polymeric Piezoelectric material); U.S. Pat. No. 6,056,694 to Watanabe (Wave Receiving Apparatus and Ultrasonic Diagnostic Apparatus); Dyson (Apparatus for Ultrasonic Tissue Investigation); U.S. Pat. No. 6,289,231 to Watanabe (Wave Receiving Apparatus and Ultrasonic Diagnostic Apparatus); U.S. Pat. No. 6,397,681 to Mizunoya (Portable Ultrasonic Detector); U.S. Pat. No. 6,641,535 to Buschke (Ultrasonic Probe, in Particular for Manual Inspection); U.S. Pat. No. 6,716,173 to Satoh (Ultrasonic Imaging Method and Ultrasonic Imaging Apparatus); and the like. The content of each of the aforementioned patents is hereby incorporated by reference into this specification.

While ultrasound transducers are described in detail herein, it should be noted that other transducers have been contemplated for use with the present invention and are considered within its scope. For example, radio waves may be adapted for use with certain embodiments of the invention. In one embodiment, the transducer is a piezoelectric transducer. In one embodiment the footplate 104 includes a sonic lens (see element 116 in FIG. 1F) in the cavity that houses the transducer crystal 103. In another embodiment the sonic lens is formed by the configuration of the footplate 104 itself. In still another embodiment the sonic lens is interposed between the transducer 103 and the bottom surface of the footplate 104. In one embodiment, a plurality of sonic lens are used, thus allowing variable focal points. For an excellent discussion of sonic lens technology, reference may be had to U.S. Pat. No. 4,399,704 to Gardineer (Ultrasound scanner having compound transceiver for multiple optimal focus), the contents of which are hereby incorporated by reference into this specification.

Referring again to FIGS. 1A and 1B, and in the embodiment depicted therein, footplate 104 is configured to be pressed against a surface, such as a patient's forearm, leg, or ribs. In the embodiment depicted, footplate 104 is filled with a coupling medium 108 which promotes the transfer of ultrasound waves from the transducer 103 to the target (not shown). Such coupling mediums are known to those skilled in the art. Coupling mediums facilitate the transfer of sonic energy from the transducer to the target, having acoustic impedance similar to that of the target. Typical coupling mediums include aqueous or water-based gels. Reference may be had to Bishop, S., Draper, D. O., Knight, K. L., Brent, F. J., & Eggett, D. (2004). Human Tissue-Temperature Rise During Ultrasound Treatments With the Aquaflex Gel Pad. J. Athl. Train., 39, 126-131.

Referring again to FIGS. 1A and 1B, and in the embodiment depicted therein, display 106 is comprised of at least one light emitting diode 110. In the embodiment depicted in FIGS. 1A and 1B, three such diodes are present. As would be apparent to one skilled in the art, a variety of display units may be used. For example, one may use a liquid crystal display, a simple light, a vibrating element, a speaker for producing sound, or any other means for notifying the user of the device 100 that a certain predetermined condition has been met. In one embodiment, display 106 includes a power button (not shown). In another embodiment, display 106 includes means for supplying information to a processor (not shown) that is housed within device 100. Display 106 is comprised a categorical display. As would be apparent to one skilled in the art, a categorical display is a display with discrete categories of indications rather than a continuum of indications, such as a display configured to project an image of the bone. One type of categorical display is a binary display, which has only two discrete categories—threshold condition met and threshold condition not met. Another type of categorical display is a series of indicators that illustrates how many threshold conditions have been met.

As depicted in FIGS. 1A and 1B, device 100 is further comprised of a processor (not shown) that is in electrical communication with ultrasound transducer 103 and display 106. The aforementioned processor controls various actions of transducer 103 and properties of the ultrasound signals it produces, such as, but not limited to; initiation and termination of the generation of ultrasound waves, control of the power of ultrasound emitted, control of the frequency of ultrasound waves emitted, processing of the electrical signal resulting from the returning ultrasound waves, and the like. Similarly, the aforementioned processor controls various properties of display 106 such as, but not limited to; the transmission of data from the processor to display 106 for observation by the user of device 100. In one embodiment, as previously discussed, display 106 is comprised of means for supplying information to the processor. In this manner, the user may alter the data contained within the processor to control the duration and repetition frequency of the voltage pulse applied to the transducer, the power of the ultrasound signal emitted, the frequency of the ultrasound signal emitted, similar parameters of the returned ultrasound signal, and a predetermined threshold condition (to be discussed in detail elsewhere in this specification) or other parameters associated with the electrical signals returning to the processor. In one embodiment, the means for supplying information to the processor is comprised of a mode selector, wherein the mode is selected from the group consisting of a calibration mode (baseline measurement), a data acquisition mode (obtaining a detection measurement), and an off mode. In another embodiment, some or all of these properties are automatically configured and/or reconfigured by the processor, thus requiring no user intervention.

FIGS. 1D and 1E are depictions of another embodiment of the present invention. FIG. 1E is an exploded view of device 114, illustrated in FIG. 1D. In the embodiment depicted in FIG. 1E, device 114 is comprised of display 106, light emitting diodes 110, probe 102, footplate 112, transducer 103 (shown exploded from probe), gel-soaked pad 118 impregnated with a coupling medium, and switch button 120.

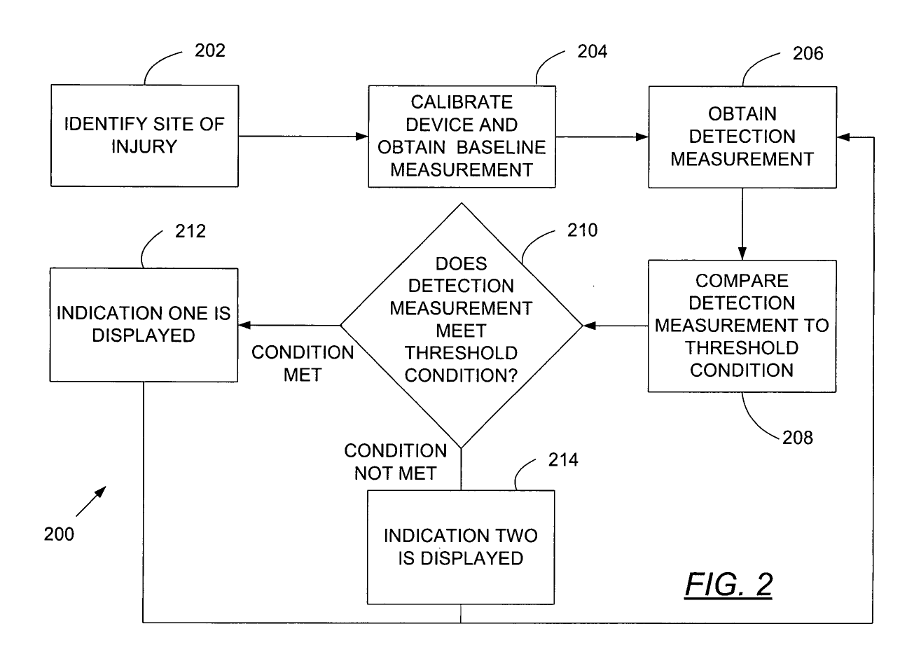

FIG. 2 is a flow diagram of one process 200 of the present invention. As illustrated in FIG. 2, in step 202, a site of injury is identified by the operator. As is known by those skilled in the art, a site of injury may be perceivably traumatized even to an unskilled observer (e.g., having obvious limb deformity, open (visible) fracture, partial amputation, etc.) or subtly traumatized (e.g., having any one or a combination of pain, swelling, tenderness, heat, redness, bruising, abrasion, etc.). It is preferred that process 200 be used for sites of injury which are subtly traumatized. For example, a patient may be suspected of having a broken forearm (e.g. ulna or radius bone), but the trauma is not so severe that a break is clearly perceptible to a medically unskilled observer. In one embodiment, to use the methods and apparatus disclosed in this application to determine if a bone is broken, a baseline measurement of an un-traumatized region of bone is first conducted. In another embodiment, a baseline measurement is not taken, and only a detection measurement is taken. In such an embodiment, the detection measurement is compared to a threshold condition stored in the processor.

Once a site of injury has been identified, the device is activated by operation of a power switch. In one embodiment, the device is battery powered. In one such embodiment, display 106 includes a “low battery” indicator, such as a light or sound. In another embodiment, the device is powered by connection to a wall outlet. Once the device is powered on, in the embodiment depicted, a self check is performed.

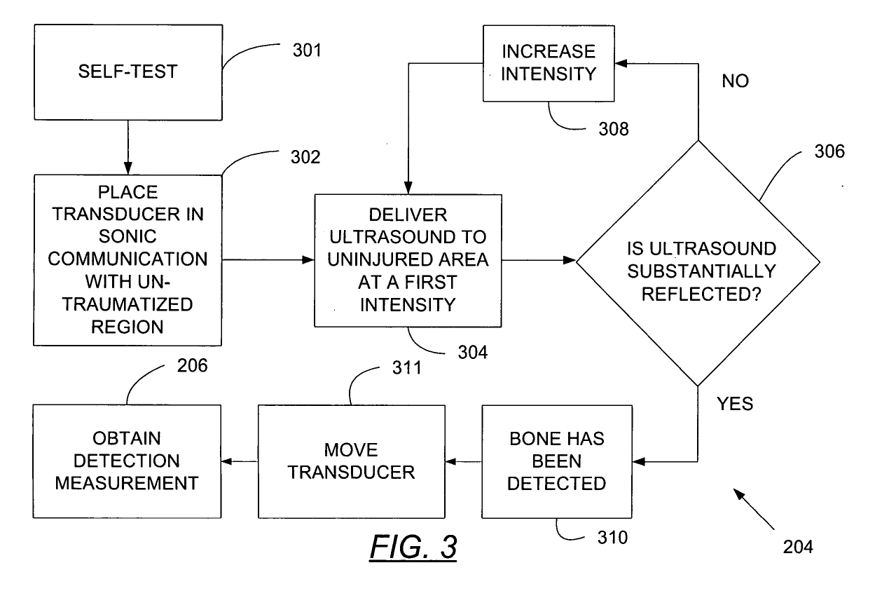

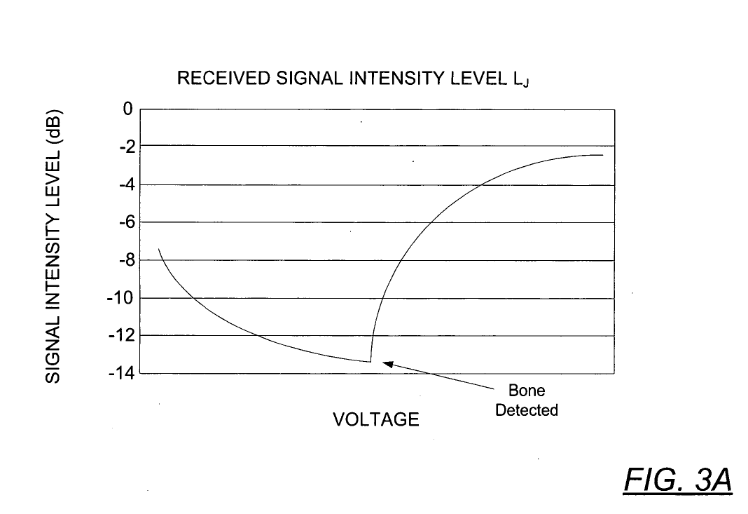

In one embodiment, and prior to or during step 204, a self-check of the device is performed. Reference may be had to FIG. 3, and step 301 illustrated therein. In such a self-check, the processor checks the device to ensure it will function properly. For example, the processor may determine if there is adequate power in the power supply or battery to conduct a scanning session, that all of the light emitting diodes or other display components are functional, and that the transducer itself is functional. In one embodiment, a sample of known composition (a “phantom”) is integrated into the device's case and functions as a suitable test material. The operator places the device in sonic communication with the phantom to determine that send and receive functions are operating normally. In one embodiment, the processor causes a green light emitting diode (LED) to illuminate if the self-test is normal, and a flashing red LED if there is a system failure. Once the device has successfully completed a self-check, the user then identifies a site of injury. Other means of self-test are not excluded by this description.

As seen in FIG. 2, and in step 202 thereof, which is optional, a baseline measurement is obtained which is indicative of an un-traumatized region of bone. As will be discussed elsewhere in this specification, this baseline measurement allows the device to accommodate for the various thickness and composition of intervening tissue that may be present between the transducer 103 (see FIGS. 1A and 1B) and the target bone. For example, such intervening matter may be adipose (fat) tissue, muscle tissue, blood vessel, and the like. A more detailed illustration of the procedures involved in step 204 may be found in FIG. 3.

FIG. 3 is a depiction of the steps involved in the execution of step 204 (obtaining a baseline measurement). In one embodiment, the step 204 is automated by the processor; i.e. the device is self-calibrating. As illustrated in FIG. 3, an optional self-check (step 301) may be performed. As illustrated in step 302 of FIG. 3, a transducer (such as transducer 103, shown in FIG. 1) is placed in sonic communication with an un-traumatized region on the patient. It is preferred that the un-traumatized region is on the same bone and adjacent to the injured region, with a small length of un-traumatized bone disposed between the placement site and the injured region. One means of ensuring that the transducer is in sonic communication with the injured area is to place the transducer directly on the un-traumatized region, thus permitting the ultrasound waves to be transmitted from the transducer to the un-traumatized region. Another means of ensuring sonic communication between the transducer and the un-traumatized region is by use of the aforementioned coupling mediums. In one embodiment a pre-fabricated and fitted gel-soaked pad is inserted by the operator into the open space on the bottom of the footplate. In another embodiment the coupling gel is introduced by the operator into the open space on the bottom of the footplate. Once the transducer is in sonic communication with the un-traumatized region, ultrasound is then delivered at a first power level.

In step 304, shown in FIG. 3, ultrasound of a predetermined power, frequency band, and direction is generated by the ultrasound transducer and transferred to the uninjured area. In one embodiment, the device is placed in calibration mode prior to the execution of step 304, thus ensuring the device will properly interpret the return signal as a calibration signal and not a data acquisition signal. Depending on the ultrasound power emitted, the ultrasound waves will penetrate the tissue to a certain depth in a substantially homogeneous medium. In one embodiment, the first power level corresponds to a voltage of lmV applied to the transducer crystal. If the resulting waves encounter bone at, or before, the certain depth, then the ultrasound waves will be substantially reflected backwards and subsequently detected by the transducer at an intensity level above a predetermined threshold. If the waves do not encounter bone before the predetermined depth is reached, then the transducer will not detect the reflected wave of interest, and merely detect wave backscattering, the intensity level of which will be below the predetermined threshold condition stored in the processor. As is known to those skilled in the art, bone is an excellent reflector of ultrasound energy. Reference may be had to The Biomedical Engineering Handbook (1995 CRC Press LLC, Joseph Bronzino Ed.) at page 1100, which states “The reflection of acoustic energy from bone is only 3 dB below that of a perfect reflector.”

In the ensuing discussion unless otherwise specified, characteristics of the electrical signal that correspond to physically real characteristics of the ultrasound signal will be referred to in terms of the ultrasound signal characteristics, for clarity. It will be understood by one skilled in the art that such correspondence is appropriate. Referring again to FIG. 3, and in step 306 depicted therein, the processor of device 100 (see FIG. 1) attempts to detect reflected ultrasonic energy. In one embodiment, the detection of the reflected ultrasound wave is based on the predetermined sound intensity level of the reflected signal. In another embodiment, the frequency spectrum or power spectrum of the returned wave is used. In yet another embodiment a signal processed according to widely-known mathematical transforms is used. When reflected signal consistent with predetermined characteristics of bone reflection is detected (see step 310), then the bone has been detected using the current ultrasound power. The user may then proceed to obtain the detection measurement (step 206 of FIG. 2 and FIG. 3) by subjecting the injured region of the bone to the waves produced by the transducer. If the reflected signal characteristic of bone reflection is not detected (see step 308), then the power of the emitted ultrasound signal is increased (see step 308) so as to effectively scan at a greater depth. In one embodiment, the intensity of the ultrasound is increased by 10 mV every time step 308 is executed. The scanning step (see step 304) is then repeated at this new intensity and greater depth. The process is repeated until a highly reflective surface (i.e. bone) is detected. In one embodiment, the process is repeated until a predetermined percentage of the emitted ultrasound signal is reflected.

In another embodiment, the device is self-calibrating. In one such embodiment, the device monitors the received signal intensity level L_{J}. As was defined above, L_{J} is a dimensionless number comprised of the log₁₀ of a ratio of a given signal intensity to a predetermined standard, expressed in dB. In one embodiment of this method, L_{J} is the log₁₀ of the ratio of the received signal intensity (Jᵣ) to the emitted signal intensity (Jₑ) (at the transducer face Jₑ is equivalent to the emitted signal power P_{AC}). As is shown in FIG. 3A, when the emitted signal is of such intensity that bone has not yet been reached, L_{J} actually decreases with each linear increase in emitted signal. This is because the returned signal increases only in proportion to the inverse square of the increased signal, so that the ratio J_{R}/J_{E} becomes smaller as J_{E} increases faster than J_{R}. When bone is detected, however, the log ratio L_{J} rapidly becomes large, as a large amount of emitted signal is reflected and received. At that point, because of the highly reflective nature of bone, for each incremental increase in emitted sound intensity there is a directly proportional increase in the received sound intensity, so that L_{J} increases rapidly with each incremental increase in emitted intensity. Thus, the self-calibrating device simply increases the power of the emitted signal until an increase in received sound intensity level (L_{J}) is obtained, at which time bone has been detected (step 310 in FIG. 3). In another embodiment, the power of the emitted signal is increased until the sound intensity level continues to increase past a pre-determined threshold condition, thereby detecting bone. In one embodiment, the device automatically switches to data acquisition mode when such a bone is detected.

The processor stores certain parameters associated with the calibration process. For example, the device may store one or more of the following parameters; ultrasound power generated, intensity of reflected ultrasound signal, time of flight of ultrasound signal, frequency and power spectrum of reflected signal, the output of various mathematical operations and transforms on the signal, and the like. In one embodiment, the device 100 remains stationary throughout the aforementioned steps.

In another embodiment, not shown, the baseline measurement is determined by first causing the transducer to emit a signal at a fixed ultrasound power sufficiently large to penetrate any reasonable thickness of soft tissue. In such an embodiment, the processor gradually decreases its sensitivity to returned echo intensity until the point that the strong bone signal intensity fails to meet a predetermined threshold condition.

In yet another embodiment, not shown, the baseline measurement is determined by causing the transducer to emit a signal at a fixed ultrasound power sufficiently large to penetrate any reasonable thickness of soft tissue. In this embodiment the processor simply identifies the echo with the largest sound intensity as bone, and establishes the other signal parameters associated with that echo (e.g., time of flight, frequency and power spectra, mathematical transforms of signal, and others) at their baseline levels. This embodiment has the advantage of simplicity, in that step 308 and the re-iteration of steps 304, 306, and 308 may be omitted. In this embodiment the determination that bone has been detected 310 and the baseline measurement of associated signal parameters 204 is accomplished in a single step.

A variety of signal characteristics may be used to determine the baseline measurement. In one embodiment, the sound intensity or sound intensity level of the signal at baseline is used by the processor as an indicator of relative signal quality, with the value at baseline defined by the processor as 100%.

Referring again to FIG. 3, and to step 311 (which is optional) depicted therein, once the proper signal intensity has been determined, the probe is moved along a short segment of the long axis of the bone adjacent to the area of suspected injury (i.e. the injured region) at a substantially constant speed and pressure. During such movement, the device is in data acquisition mode (i.e. the device is obtaining a detection measurement as in step 206).

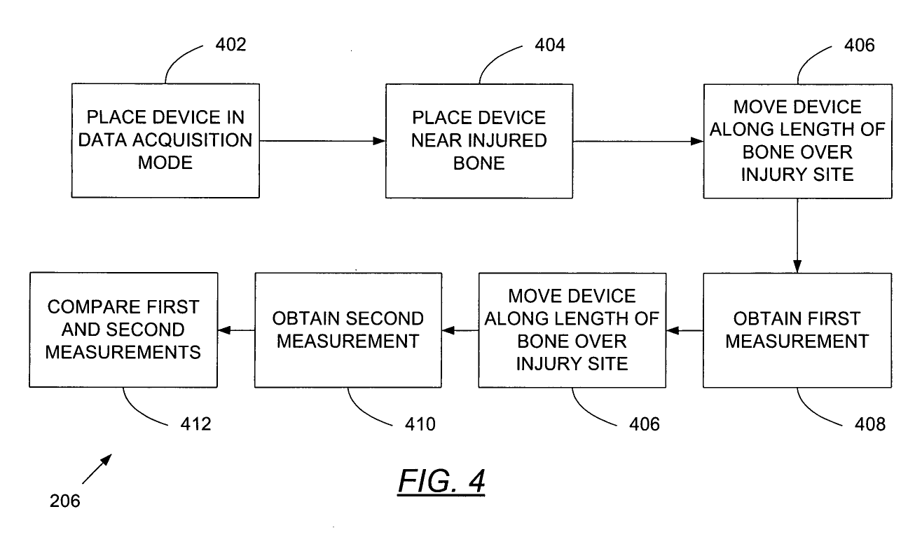

Referring again to FIG. 2, and process 200 depicted therein, once the baseline measurement has been obtained in step 204, a detection measurement is obtained in step 206 of process 200, depicted in FIG. 2. A detection measurement is obtained by subjecting the bone to the waves produced by the transducer. The processor monitors the reflected signal and compares this signal to a threshold condition stored in the processor. Based on this comparison, the processor decides which actions are to follow (step 210). If the reflected signal meets the predetermined threshold condition stored in the processor then the device continues to obtain the detection measurement (returns to step 206) and the display 106 (see FIG. 1) generates a first indication (step 212). If the reflected signal does not meet the threshold condition then the display 106 generates a second indication (step 214) and continues the detection measurement (returns to step 206). In one embodiment, the first indication corresponds to a broken bone being detected and the second indication corresponds to no break in the bone being detected. In another embodiment, the first indication corresponds to no break in the bone being detected and the second indication corresponds to a break in the bone being detected. In another embodiment, not shown, process 200 is further comprised of the step of the processor resetting the threshold condition while obtaining the detection measurement or baseline measurement based on an analysis of such measurement. A more detailed illustration of the procedures involved in certain embodiments of step 206 may be found in FIG. 4.

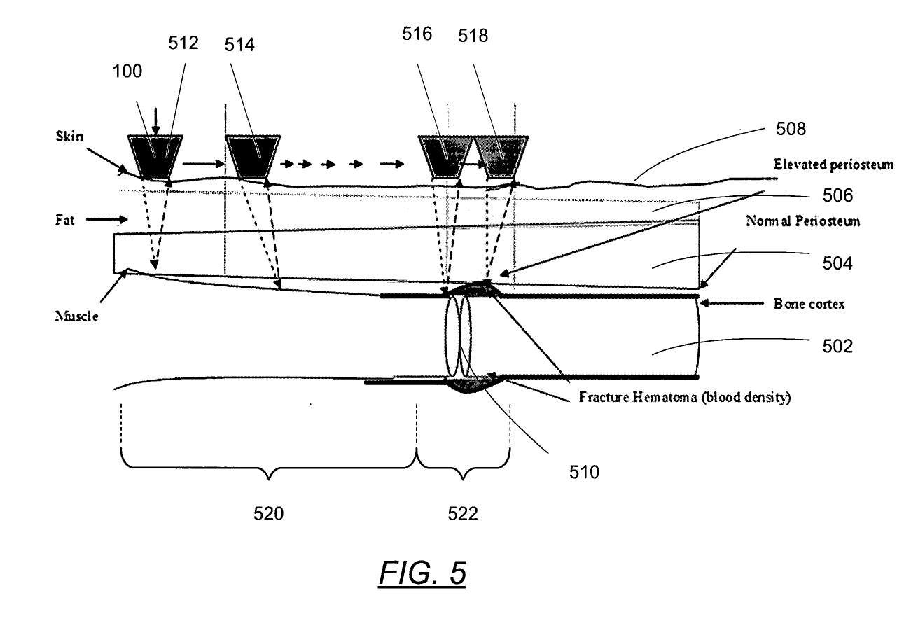

FIG. 4 is a more detailed depiction of the steps involved in the execution of one embodiment of step 206 (obtaining a detection measurement). The steps described in FIG. 4 will be described with reference to FIG. 5.

In step 402 (which is optional) of process 206, illustrated in FIG. 4, the device is switched from calibration mode to data acquisition mode (i.e. detection measurement mode). This ensures the device will properly interpret a returning ultrasound signal as a data signal, and not a calibration signal. In one embodiment, the device stores the various measured and calculated signal characteristics that were determined during the calibration process and analyzes such characteristics of the baseline measurement to determine and set the aforementioned threshold condition. In one embodiment, the device automatically switches from calibration mode to data acquisition mode when bone is detected (step 310 illustrated in FIG. 3). In another embodiment the devices automatically switches from calibration mode to data acquisition mode when movement is detected resulting from a substantial change in signal characteristics from baseline. So long as no change, or change below a predetermined threshold of variation, is detected, the processor will determine that the probe is not in motion and notify the user that the device has successfully acquired a baseline measurement. One means for notification may be, for example, the illumination of one or more light emitting diodes.

Referring again to step 404 of process 206, illustrated in FIG. 4, and with further reference to FIG. 5, the device 100 is placed near the site of injury (position 516). In one embodiment, the site of placement (position 514) is between the site of calibration (position 512) and the injured region (position 516). In another embodiment, the site of placement is adjacent to the site of injury. Reference may be had to FIG. 5. Once the device 100 has been properly positioned, the device begins to emit ultrasound waves of a predetermined power. This power may be selected, at least in part, by the parameters that were determined during the calibration step. In another embodiment, the power emitted by the transducer may be fixed.

Referring again to FIG. 4 and step 406 therein, the device is moved along at least a portion of the length of the un-traumatized region towards and across the suspected injury region while continuing to emit ultrasound waves. The processor begins to take multiple, repeated measures at a rate that may vary between 0.5 to 10 kHz. In the embodiment illustrated in FIG. 4, a first measurement is taken in step 408. Thereafter, step 406 is repeated and the device is moved further along the length of bone. A second measurement is then taken (step 410). The processor compares the signal parameter(s) at each point “n” in time (first measurement) with the value(s) at the preceding point, “n−1,” (second measurement) measured during the preceding measurement cycle. A comparison of the first and second measurement is then made (step 412). So long as no change, or change below a predetermined threshold of variation, is detected, the processor will determine that no anomaly has been detected and notify the user that the scan may continue. One means for notification may be, for example, the illumination of one or more light emitting diodes.

A variety of observed properties of the reflected signal may be processed in this manner or in a similar manner. By way of illustration, and not limitation, these observed properties include the amplitude of the returned signal at a specified wavelength, the wavelength of the returned signal where the maximum amplitude occurs (i.e. a returned peak frequency or maximum), an area under the spectral curve of the returned signal, mathematical derivatives of any of the observed properties, and combinations thereof. An appropriate threshold condition is set according to which property is being observed.

In one embodiment, the second derivative of the returned amplitude at a specified wavelength is calculated as a function of time and monitored by the processor. In another embodiment, the derivative as a function of distance moved is calculated. In one such embodiment, the threshold condition is “greater than or equal to X” where X is a number. The second derivative of the returned amplitude is compared to this threshold condition to determine which of the indicators should be generated.

In another embodiment, the threshold condition is a threshold region with an upper and lower value. In one such embodiment, the threshold region is “greater than X, but less than Y” where X and Y are numbers that define the range of the region.

In yet another embodiment, the threshold condition is a distribution width threshold condition. In one such embodiment, the distribution width is “less than or equal to X” where X is a number. The reflected signal is monitored and its power spectrum is analyzed to determine its returned peak frequency. The shape of the curve of this power spectrum is analyzed to determine its width. In one embodiment, the width at half the height of the returned peak frequency is measured. This width is compared to the distribution width threshold condition for compliance with such condition.

During the detection measurement, signal quality may deteriorate if the operator allows the transducer to drift out of alignment with the long axis of the bone in question. To minimize this potential source of error the processor will, in one embodiment, cause a “poor signal” alarm to sound, or alternatively cause a change in the visual display, alerting the operator should the sound intensity level (L_{J}) of the received bone signal fall below a predetermined value. In the event of the “poor signal” alarm being activated, the operating instructions will specify that the operator move the device in a direction substantially perpendicular to the long axis of the bone until the alarm is extinguished. In most cases it will be readily apparent to the operator in which direction the device should be moved. In the case in which such direction is not readily apparent, the operating instructions will specify that the operator first move the footplate in one direction and if the poor signal warning is not extinguished readily, then the operator will move the device in the opposite direction.

FIG. 5 is an illustration of the detection of a bone fracture using one embodiment of the present invention. As seen in FIG. 5, the bone 502 is comprised of a fracture 510 present in injured region 522. Also shown in FIG. 5 is un-traumatized region 520. Disposed above bone 502 is a layer of muscle 504, a layer of fat 506, and the layer of skin 508. Disposed on the layer of skin 508 is device 100. Device 100 is shown in several positions along the length of bone 502, such as position 512, position 514, position 516, and position 518.

In one embodiment, which is preferred, there is a single site of initial placement. In such an embodiment, the device 100 is not removed from the skin until the entire process is complete. For example, and with reference to FIG. 5, the device 100 is placed at position 512 and the calibration is performed (step 204). Thereafter, and without removing the device 100 from the surface, the device is placed in data acquisition mode and is thereafter moved along the length of the bone to position 514, and thereafter to position 516. In one embodiment, during such movement, the device continues to emit, receive, and process ultrasound signals during such movement.

With reference to FIG. 5, the device 100 is moved from the site of placement (position 514), along the length of bone 510, to position 516, and eventually position 518. It is preferred that such movement has a substantially steady rate. During such movement, ultrasound waves are continually or intermittently generated by device 100, reflected off of bone 510, and subsequently detected by device 100. Comparison of certain characteristics of the reflected signal to the threshold condition allows the device 100 to indicate if a fracture has been detected.

As is illustrated in FIG. 5, the site of scanning is not necessarily homogeneous. For example, it is clear that the topography of normal bone 510 is irregular. Furthermore, it is clear that the layer of muscle 504, the layer of fat 506, and the layer of skin 508 vary in depth over the scan area. In one embodiment, the device 100 compensates for such variations by plotting the return signal and taking the second derivative of the values of each characteristic of the signal with regard to time or distance. In such an embodiment, the fracture is detected by observing the acceleration of change of the tissue morphology. If the acceleration of change (the second order derivative) exceeds a certain value, then a signal is generated by the device that indicates a high likelihood of bone fracture. Gradual changes, such as those characteristic of healthy and intact tissue, however, fall below the certain value, and thus do not generate such a signal. In one embodiment, the predetermined value is selectable by the user.

In one embodiment, an “overpressure alarm” is built into the device to notify the user that excessive pressure is being applied. In another embodiment, an “underpressure alarm” is used. Such alarms may be based upon the pressure applied by the footplate to the skin. Alternatively or additionally, such alarms may be based on a characteristic of the signal dropping below a predetermined threshold.

In one embodiment, the processor automatically detects if the device is in motion, and thus automatically causes the device to switch from calibration mode to data acquisition mode. In one embodiment, the device automatically determines that it should be in calibration mode when the magnitude of a characteristic of the reflected signal is substantially unchanging. Likewise, in another embodiment, the device automatically determines it should be in data acquisition mode when the magnitude of a characteristic of the reflected signal is substantially changing.

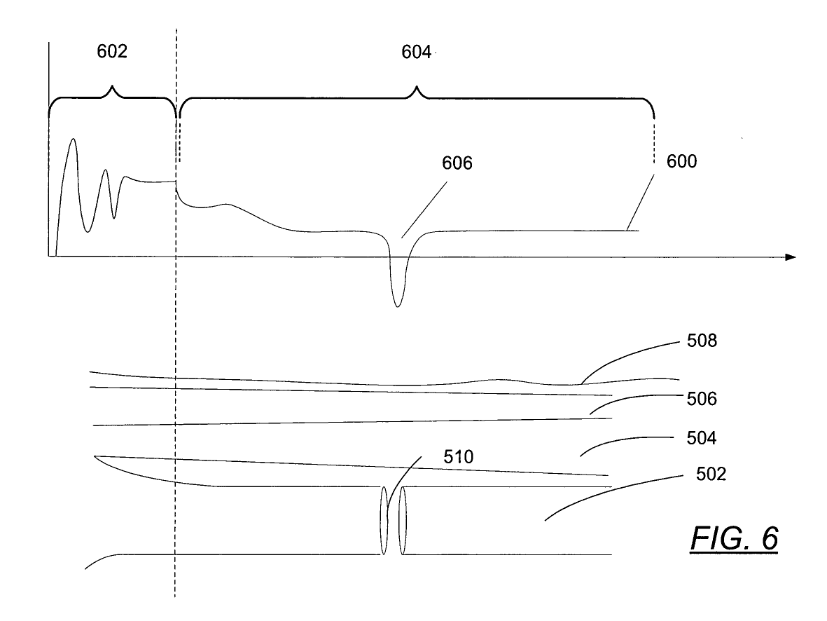

FIG. 6 is an illustration of a first derivative plot of the return signal. In one embodiment, the Y axis is signal intensity and the X axis is time. In another embodiment, the Y axis is signal intensity and the X axis is distance the probe has moved. In still another embodiment the Y axis represents at least one of time of flight and various values selected from a Fast Fourier Transform, a discrete Gabor Transform, a discrete Zak transform, and a combination of these or other mathematical signal operations. As illustrated in FIG. 6, signal 600 is comprised of calibration region 602 and data acquisition region 604. When the device detects fracture 510, a signal deviation 606 is seen. In the embodiment depicted, signal 606 has a negative deviation. Such a signal may result, for example, by a fracture in a bone. Other signal deviations are possible.

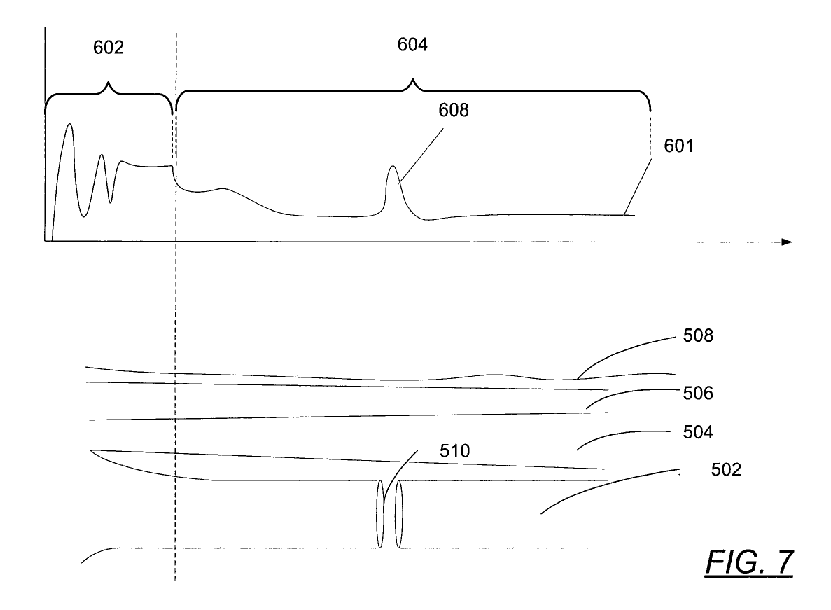

FIG. 7 is an illustration of another deviation 608 of signal 601. In the embodiment depicted in FIG. 7, the signal deviation has a positive direction. Such a signal may result, for example, by a hematoma surrounding a fracture, or from a displacement of bone towards the transducer such as occurs in a displaced fracture or a “crumple zone” of compression or buckle fracture. See, for example, FIG. 5.

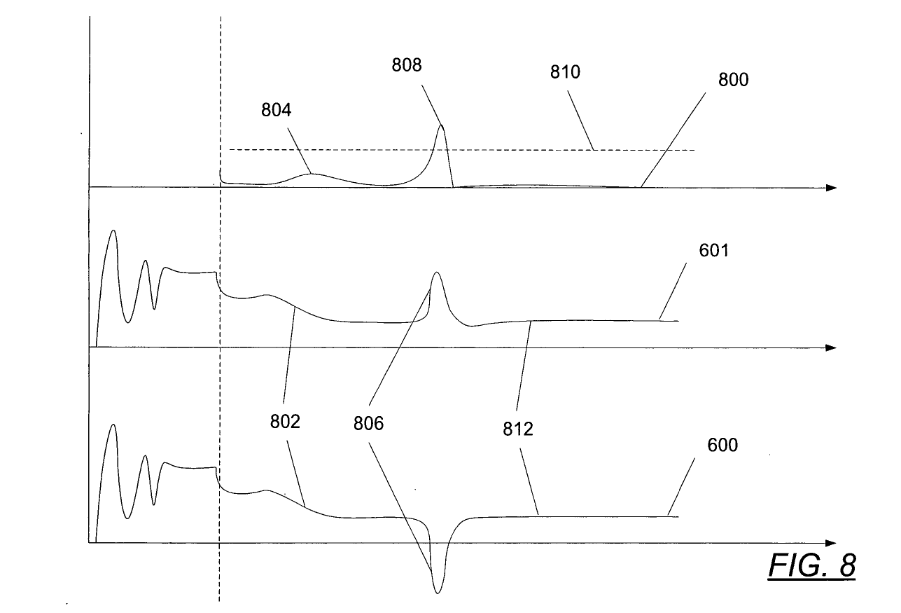

FIG. 8 is an illustration of three signals of the present invention. Signal 600 and signal 601 are comprised of deviation 806 and normal variation 802. In the embodiment depicted, signal 600 differs from signal 601 in that the sign of the deviation is negative. Signal 800 is the absolute value of a second derivative of the aforementioned signal (|[Δₙ−Δₙ₋₁]|=|Δ*ₙ|). Since signal 800 is based on the absolute value of the signal, both signal 600 and signal 601 result in substantially the same second derivative plot.

Signal 800 of FIG. 8 represents the acceleration of change of the signal. For example, in both signal 600 and signal 601, the signal is slowly changing at normal variation 802. Such a slow change results in peak 804. It is noteworthy that the acceleration of the signal change (normal variation 802) is of such a magnitude that peak 804 remains below threshold 810. In such an embodiment, the threshold condition is not met when peak 804 is less than threshold 810. In contrast, the signal 600 and signal 601 both change rapidly at aberration 806. Such a rapid change results in peak 808. Peak 808 has a magnitude that exceeds threshold 810. In one embodiment, peaks which exceed the threshold 810 cause the device to generate a signal that is perceptible to the user (referred to herein as the first or second indication, for example a light, a sound, a vibration, etc.) Similarly, plateau 812 has substantially no change in the signal. As such, no corresponding peak is generated in the second derivative plot and the remaining indication (either the first or second indication) is displayed. It should be clear from the previous discussion that if the first indication corresponds to the threshold condition being met, then the second indication corresponds to the threshold condition not being met. Similarly, if the first indication corresponds to the threshold condition not being met, then the second indication corresponds to the threshold being met. The first and second indication may be any indication that communications the threshold condition state to the user. For example, the first indication may be a light being activated, and the second indication is the same light not being activated.

In one embodiment, the device has a single threshold setting stored in the processor. In one embodiment, this threshold may be configured by the user by operation of the means for supplying information to a processor in display 106. When the peak of the second derivative plot meets the threshold condition, then a light, such as light emitting diode 110 is activated.

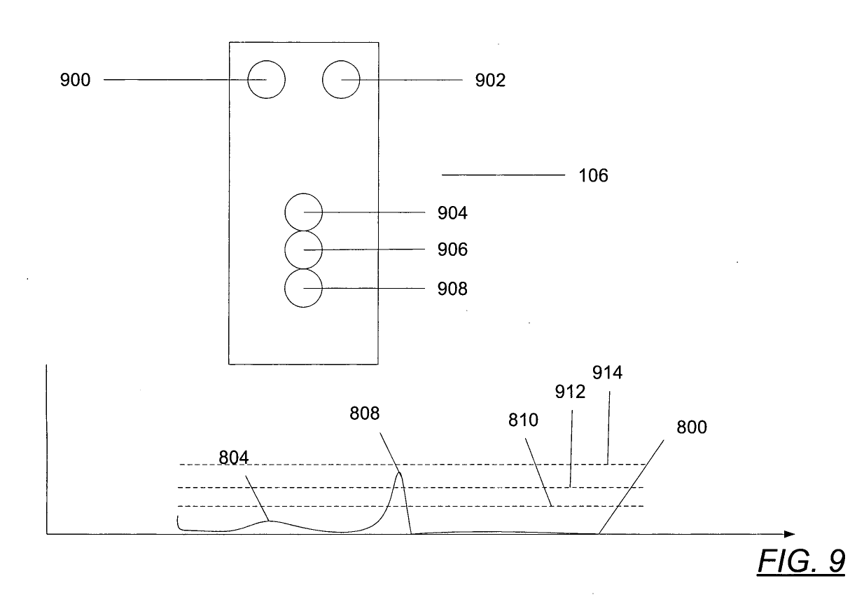

In another embodiment, the device has at least two threshold conditions stored in the processor. In one embodiment, the reflected signal must satisfy at least two of the threshold conditions before the first and/or second indications are displayed. It is clear that any number of thresholds may be present. Reference may be had to FIG. 9.

In the embodiment depicted in FIG. 9 three such threshold conditions (810, 912, and 914) are present. In one embodiment, these threshold conditions may be configured by the user. In another embodiment, the threshold conditions are preprogrammed into the processor and are not configured by the user. If the peak of the second derivative plot meets one of the threshold conditions (812), then a first light (908 on display 106 illustrated in FIG. 9) is triggered. If the peak meets two of the threshold conditions (812 and 912), then a second light is triggered (thus lights, 908 and 906 are triggered). If a peak meets all three of the threshold conditions (812, 912 and 914), then a third light is triggered (lights, 908, 906, and 904). In the embodiment depicted, peak 808 causes lights 906 and 908 to light, but does not cause light 904 to light. In the embodiment depicted in FIG. 9, display 106 is comprised of mode selector buttons 900 and 902. Mode selector button 900, when depressed, places the device in calibration mode. Mode selector button 902, when depressed, places the device in data acquisition mode. In another embodiment, display 106 is further comprised of means to configure the threshold conditions associated with lights 904, 906, and 908. In another embodiment, the mode selection is automated. In another embodiment, the threshold condition(s) are set by the processor after analyzing the reflected signal during the baseline measurement.

Alternatively, or additionally, only a single light is present, but the color of the light indicates the number of threshold conditions that have been exceeded. For example, of no threshold condition has been met, the light is green. If a single threshold condition has been met, then the light is yellow. If two threshold conditions have been met, then the light is red. In another embodiment, there are a plurality of lights, and the lights are colored coded. For example, and with reference to FIG. 9, light 908 is green, light 906 is yellow, and light 904 is red.

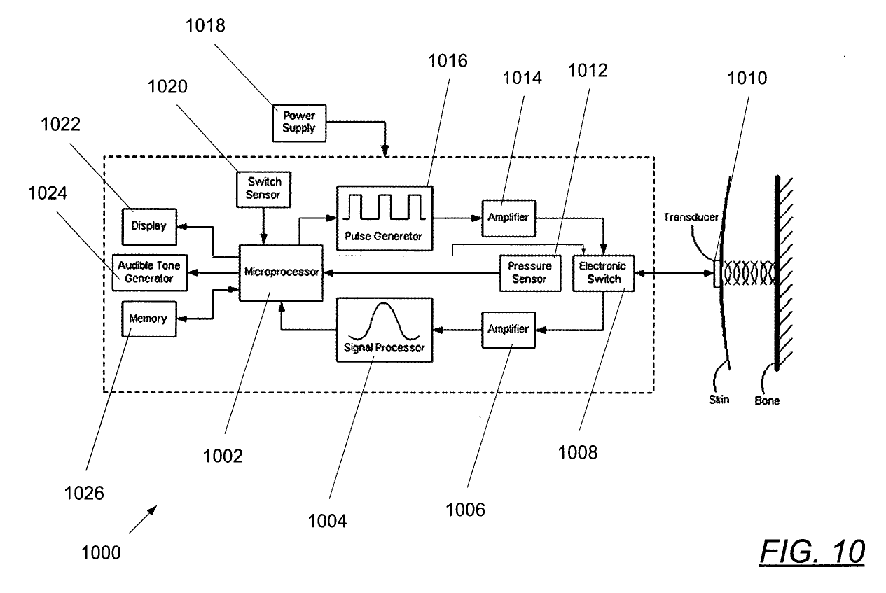

FIG. 10 is a schematic diagram of assembly 1000 which is comprised of processor 1002, signal processor 1004, amplifier 1006, switch 1008, transducer 1010, pressure sensor 1012, amplifier 1014, pulse generator 1016, power supply 1018, switch 1020, display 1022, audible tone generator 1024, and memory 1026. In the embodiment shown in FIG. 10, the transducer 1010 is a single component transducer.

In the embodiment depicted in FIG. 10, the processor 1002 controls the pulse generator 1016 so as to generate a voltage that is transferred to amplifier 1014. The voltage generated has a pattern such that certain ultrasonic waves with certain properties are generated. For example, the voltage may control the frequency and/or power of the emitted waves. The transducer is configured to both emit and receive ultrasonic waves, and the electronic switch activates either the transmission or the reception side of the circuit at rates controlled by the processor. The received waves are transformed into electrical impulses and transferred to amplifier 1006, and then to signal processor 1004, for eventual transmission back to processor 1002. Processor 1002 is in electrical communication with pressure sensor 1012, which monitors the applied pressure (i.e. overpressure and underpressure alarms that are discussed elsewhere in this specification). The processor is also in communication with switch 1020 which controls a variety of processor parameters, such as, for example, power, mode selection, and the like. The processor is also electrically connected to display 1022, audible tone generator 1024, and memory 1025. In one embodiment, memory 1026 is random access memory (RAM). In another embodiment, memory 1026 is read-only memory (ROM). Assembly 1000 is also comprised of power supply 1018. In one embodiment, power supply 1018 is a battery. In another embodiment, power supply 1018 is an AC power source such as a wall outlet.

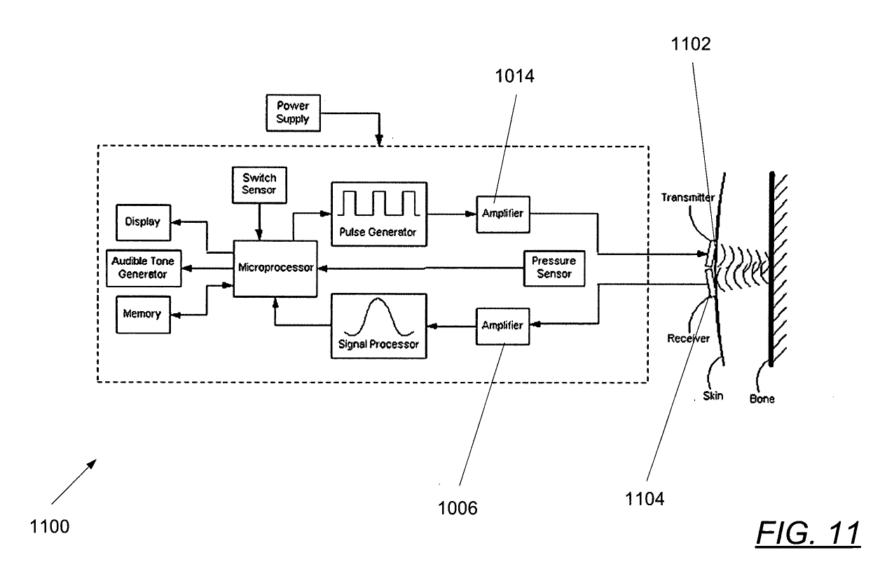

FIG. 11 is a schematic diagram of another assembly 1100 of the present invention. Assembly 1100 is substantially identical to assembly 1000 depicted in FIG. 10, except in that a duel component transducer is used. As illustrated in FIG. 11, a transmitter transducer 1102 receives electrical impulses from amplifier 1014 and transforms the impulses into ultrasound waves. Receiver transducer 1044 receives the reflected ultrasound waves and transforms the waves into electrical impulses, which are then transferred to amplifier 1006.

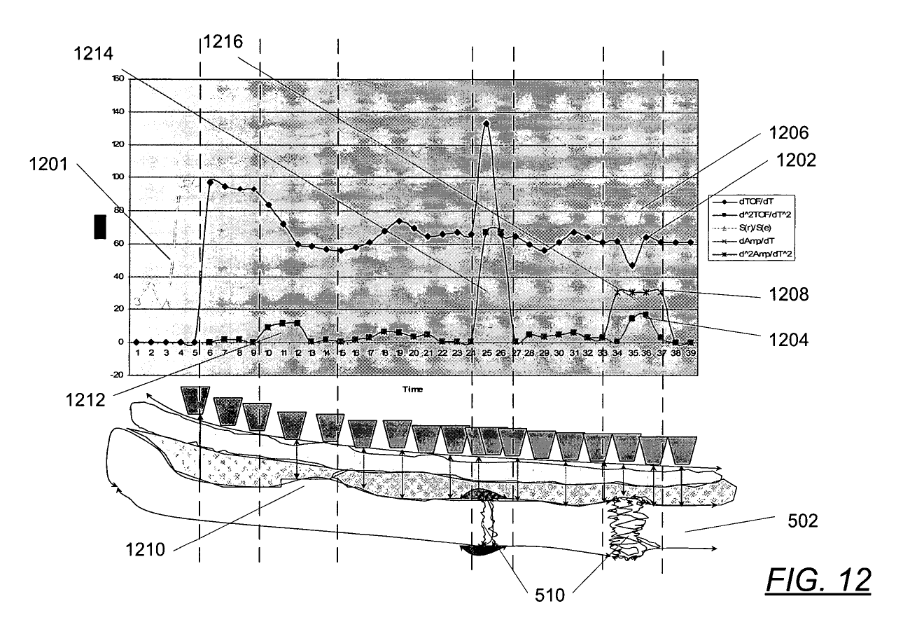

FIG. 12 is a plot and schematic diagram of a fractured bone and a plot of ultrasound signal intensity versus time. Signal 1202 is the first derivative of the signal based on the time of flight of the ultrasound wave as a function of time. Similarly, signal 1204 is the second derivative of the time of flight signal. Signal 1206 is the first derivative of the signal based on the intensity of the received ultrasound wave as a function of time. Likewise, signal 1208 is the second order derivative of the intensity signal. Signal 1201 is the sound intensity level L_{J} described above and shown in greater detail in FIG. 3A. The value of L_{J} becomes large when the received signal increases due to bone being detected.

As illustrated in FIG. 12, bone 502 is comprised of fractures 510 and 511, and natural projection 1210. Fracture 510 represents a slightly displaced fracture with physical space between the fragments and a fracture hematoma surrounding it. Fracture 511 represents a compression, “buckle,” or “torus” fracture, in which the cortex of the bone is both irregularly crumpled and displaced towards the skin. Referring to the second order derivative plots 1204 and 1208 of FIG. 12, it is clear that natural projection 1210 resulted in a small signal 1212. In contrast, fractures 510 and 511 resulted in large signals 1214 and 1216. An inherent advantage of this method is that both fracture 510, for which signal change is in the same direction for both measured signals, and fracture 511, in which signal amplitude changes negatively, but time of flight changes positively, result in correct identification as aberrant regions of bone. Conversely, although the absolute change in both signals is relatively large over normal bone projection 1210, neither second derivative signal reaches a predetermined threshold. These features of the presently disclosed method increase its ability to detect true fractures (giving the method high sensitivity) and to avoid false detection of normal bone variation as aberrancies (giving the method high specificity). As would be apparent to one skilled in the art, other mathematical operations may be performed on the signals to obtain other plots with substantially similar results.

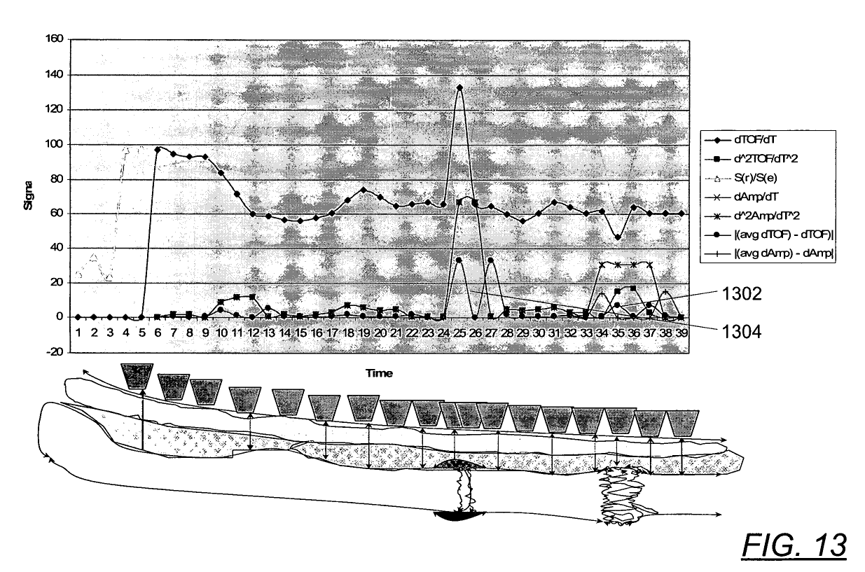

FIG. 13 is an illustration of one such alternate plot obtained by performing another mathematical operation. FIG. 13 is substantially identical to FIG. 12 except in that the plot of FIG. 13 is comprised of two additional signals; signal 1302 and signal 1304. Signal 1302 is obtained by taking the difference between the current second derivative of the time of flight signal and the average of the previous two second order time of flight signals. Similarly, signal 1304 was obtained by taking the difference between the current second order amplitude and the average of the previously two second order amplitude signals. The use of the Fast Fourier Transform, the discrete Gabor transform, the discrete Zak transform, and other similar means of manipulating raw signals may be used to provide an additional range of signals for analysis and detection of differences between normally varying regions of bone and those with sharp variations that indicate aberrancies.

### EXAMPLE 1

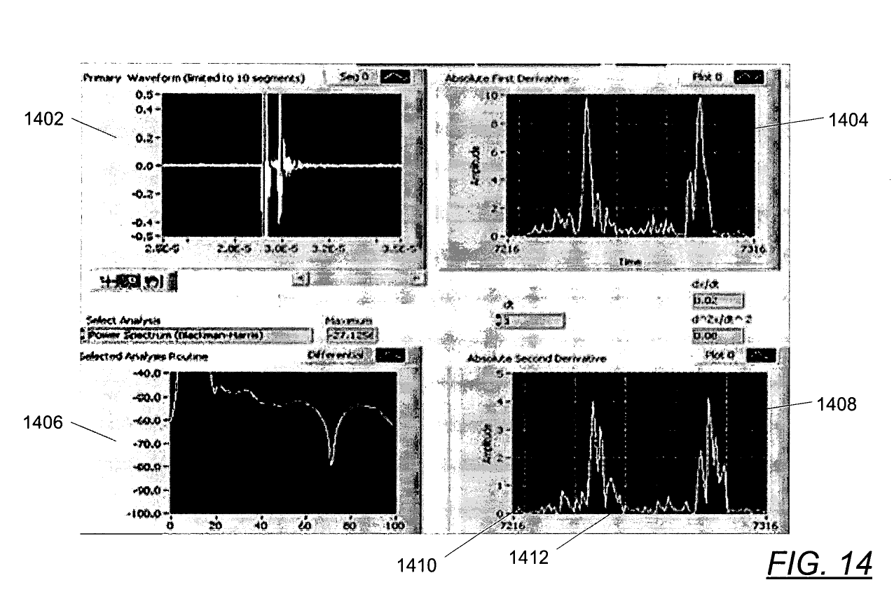

Two calcium impregnated tiles were placed next to one another such that a gap of approximately 5 mm was present between such tiles. This gap was then filed with Aquaflex brand ultrasound gel pad. Additional gel was placed over the tiles such that a substantially flat surface of gel was present over both tiles as well as the gap. Aquasonic brand coupling gel was placed over this surface. A Panametrics-NDT 20 MHz, 0.125″ ultrasonic transducer was placed in contact with the surface of the gel over the tile and moved from the starting tile, over the gap, and over the second tile. A JSR DPR300 Ultrasonic Pulser/Receiver was used to control the transducer. The received signal was transmitted from the transducer to a personal computer with the assistance of a DP308 Digitizer PCI interface card available from Acqiris. The results of this experiment are shown in FIG. 14.

As shown in FIG. 14, primary waveform 1402 is the reflected ultrasonic signal currently being sensed by the ultrasonic transducer. Graph 1406 is a spectrum of primary waveform 1402 showing the frequencies that make up the primary waveform. As is apparent, such a spectrum has a maximum wavelength. Absolute first derivative 1404 shows the history of the first derivative of this maximum wavelength as a function of time. Similarly, second derivative 1408 shows the history of the second derivative of the maximum wavelength. As the transducer moved across the starting tile and passed over the edge of the gap, peak 1410 was generated. When the transducer was moved over the edge of the gap and passed over the trailing tile, peak 1412 was generated. In this manner, the break in the calcium impregnated tiles was detected.

### EXAMPLE 2

An artificial bone manufactured by Sawbones was encased in Blue Phantom brand gel 1504. This gel is designed to closely approximate the average ultrasonic characteristics of human flesh. X-ray image 1506 shows an image of the bone 1508, an image of the gel 1504A, and an image of the bone fracture 1510. Ultrasonic transducer 1502 was placed on the surface of gel 1504 after coating gel 1504 with coupling medium (not shown). When the probe is placed over un-traumatized region 1512, a first signal was generated. When the probe is placed over traumatized region 1514, a second signal was generated. The frequency of the maximum return signal varied between approximately 9 and 10 MHz while the transducer was over un-traumatized region 1512. The frequency of the maximum return signal was consistently greater than 11.5 MHz while the transducer was disposed over traumatized region 1514. The threshold condition in the test device was configured such that that a maximum return signal less than 11 MHz resulted in a first indication on the conditional display being given over un-traumatized region 1512 and the second indication being given when the maximum return signal was greater than 11 MHz, corresponding with the transducer positioned over traumatized region 1514.

It is therefore, apparent that there has been provided, in accordance with the present invention, a method and apparatus for the detection of a bone fracture using ultrasound. While this invention has been described in conjunction with preferred embodiments thereof, it is evident that many alternatives, modifications, and variations will be apparent to those skilled in the art.

1. An apparatus for detecting a condition of a bone comprising:
a. a processor, a display and a transducer for producing waves directed to a bone for reflection of said waves, thereby producing a reflected signal, wherein said bone is comprised of an un-traumatized region and an injured region;
b. said transducer is configured to receive said reflected signal thus obtaining a detection measurement;
c. monitoring said reflected signal obtained during said step of obtaining a detection measurement with said processor,
d. comparing said reflected signal to a threshold condition stored in said processor; and
e. displaying a condition of said bone by producing a first indication on said display when said reflected signal does not meet said threshold condition, and producing a second indication on said display when said reflected signal does meet said threshold condition.

2. The apparatus as recited in claim 1, wherein said waves are ultrasonic waves.

3. The apparatus as recited in claim 2, wherein said display is a categorical display.

4. The apparatus as recited in claim 3, wherein said categorical display is a binary categorical display configured to display two states selected from the group consisting of said first indication and said second indication.

5. The apparatus as recited in claim 1, wherein said display consists of said first indication and said second indication.

6. The apparatus as recited in claim 3, wherein said apparatus does not display an image of said bone.

7. The apparatus as recited claim 3, wherein said processor calculates a derivative of said reflected signal.

8. The apparatus as recited in claim 3, wherein said transducer is a phased array transducer configured to be disposed over said bone such that at least a portion of said phased array transducer is disposed over said un-traumatized region and at least a portion of said phased array transducer is disposed over said injured region.

9. A method for detecting a condition of a bone comprising the steps of
a. disposing an apparatus over a bone, wherein said bone is comprised of an un-traumatized region and an injured region and said apparatus is comprised of a transducer for producing ultrasonic waves, a processor, and a display, and wherein said transducer is configured to receive a reflected signal that is produced when said waves reflect off said bone;
b. obtaining a detection measurement by subjecting said injured region to said waves and producing said reflected signal;
c. monitoring said reflected signal obtained during said step of obtaining a detection measurement with said processor;
d. comparing said reflected signal to a threshold condition stored in said processor; and
e. displaying a condition of said bone by producing a first indication on said display when said reflected signal does not meet said threshold condition, and producing a second indication on said display when said reflected signal does meet said threshold condition, wherein said display is a categorical display configured to display two states selected from the group consisting of said first indication and said second indication.

10. The method as recited in claim 9, further comprising the steps of
a. obtaining a baseline measurement by disposing said apparatus over said un-traumatized region, and subjecting said un-traumatized region to said waves and producing a baseline reflected signal;
b. analyzing said baseline reflected signal and setting said threshold condition based on said analysis.

11. The method as recited in claim 9, wherein said threshold condition is comprised of a threshold region with a first threshold value and a second threshold value, wherein
a. said reflected signal meets said threshold condition if said reflected signal is greater than or equal to said first threshold value and is less than or equal to said second threshold value;
b. said reflected signal does not meet said threshold condition if said reflected signal is less than said first threshold value;
c. said reflected signal does not meet said threshold condition if said reflected signal is greater than said second threshold value.

12. The method as recited in claim 9, wherein said reflected signal has an observed property selected from the group consisting of a returned amplitude, a returned peak frequency, an area under the spectral curve, derivatives of said observed properties, and combinations thereof.

13. The method as recited in claim 9, further comprising the steps of
a. calculating a derivative of said reflected signal; and
b. said threshold condition is comprised of a derivative threshold condition, wherein said first indication is produced when said derivative of said reflected signal meets said derivative threshold condition and said second indication is produced when said reflected signal does not meet said derivative threshold condition.

14. The method as recited in claim 9, wherein said reflected signal is comprised of a returned amplitude and said threshold condition is comprised of a returned amplitude threshold condition, wherein said first indication is produced when said returned amplitude meets said returned amplitude threshold condition and said second indication is produced when said returned amplitude does not meet said returned amplitude threshold condition.

15. The method as recited in claim 9, wherein said reflected signal from said injured region is comprised of a returned power spectrum with a returned peak frequency and said threshold condition is comprised of a returned peak frequency threshold condition, such that said first indication is produced when said returned peak frequency meets said returned peak frequency threshold condition and said second indication is produced when said returned peak frequency does not meet said returned peak frequency threshold condition.

16. The method as recited in claim 10, wherein both said baseline measurement and said detection measurement are obtained without moving said apparatus by selectively activating ultrasonic transducers in a predetermined order within said phased array transducer.

17. The method as recited in claim 10, further comprising the step of moving said apparatus after obtaining said baseline measurement, and prior to obtaining said detection measurement such that said apparatus is disposed over said injured area prior to said step of obtaining said detection measurement.

18. The method as recited in claim 9, wherein said reflected signal from said injured region is comprised of a plurality of wavelengths that produce a spectrum with an area under the curve of said spectrum and said threshold condition is comprised of an area threshold condition, wherein said first indication is produced when said area under the curve meets said area threshold condition and said second indication is produced when said area under the curve does not meet said area threshold condition.

19. The method as recited in claim 9, wherein said reflected signal from said injured region is comprised of a power spectrum with a distribution of returned frequencies about a returned peak frequency, and said threshold condition is comprised of a distribution width threshold condition wherein said first indication is produced when the width of said distribution of returned frequencies is less than said, distribution width threshold and said second indication is produced when the width of said distribution of returned frequencies is greater than said distribution width threshold.

20. The method as recited in claim 9, wherein said threshold condition is comprised of a first condition and a second condition, and wherein said second indication is produced when both said first condition and said second condition are met.
